(19) United States
(12) Patent Application Publication
(10) Pub. No.: US 2025/0058313 A1
(43) Pub. Date: Feb. 20, 2025

(54) LATERAL FLOW ASSAY DEVICE ADAPTED FOR BEVERAGE TESTING

(71) Applicant: KnoNap LLC, Scottsdale, AZ (US)

(72) Inventors: Danya Sherman, Arlington, VA (US); Megan McLarty, San Diego, CA (US); Michael Hughes, Escondido, CA (US)

(21) Appl. No.: 18/808,593

(22) Filed: Aug. 19, 2024

Related U.S. Application Data
(60) Provisional application No. 63/533,405, filed on Aug. 18, 2023.

(51) Int. Cl. B01L 3/00; G01N 33/14

(52) U.S. Cl. CPC B01L 3/5023; G01N 33/146; B01L 2300/0681; B01L 2300/069; B01L 2300/0825; B01L 2300/0887; B01L 2300/12; B01L 2300/126

(57) ABSTRACT

Devices are disclosed for the detection of illicit substances within beverages. A lateral flow assay device for testing beverages comprises a backing card, a plurality of pads adhered to the backing card, the plurality of pads comprising at least a sample pad, a stacking pad, and a conjugate pad, wherein the stacking pad has a first end and a second end, the stacking pad is overlapped by the sample pad by a first length at the first end, and the stacking pad overlaps the conjugate pad by a second length at the second end.

### RELATED APPLICATION(S)

This application claims the benefit of priority to U.S. Provisional Application No. 63/533,405, filed Aug. 18, 2023, which is incorporated herein by reference in its entirety.

### BACKGROUND

The disclosure is generally directed to devices for screening beverages, and more specifically, to lateral flow assay devices adapted for testing alcoholic beverage screening for surreptitious drugs such as ketamine and benzodiazepines.

### SUMMARY

According to embodiments of the present disclosure, rapid assay devices for detecting benzodiazepines or other drugs are provided. In various embodiments, a lateral flow assay test device comprises a backing card and a plurality of pads adhered to the backing card.

In one embodiment, a lateral flow assay device for testing beverages, the lateral flow assay device comprising a backing card, a plurality of pads adhered to the backing card, the plurality of pads comprising at least a sample pad, a stacking pad, and a conjugate pad, wherein the stacking pad has a first end and a second end, the stacking pad is overlapped by the sample pad by a first length at the first end, and the stacking pad overlaps the conjugate pad by a second length at the second end.

In some embodiments, the plurality of pads forms a continuous flow path along the backing card. In some embodiments, each pad of the plurality of pads is in fluid communication with an overlapping pad of the plurality of pads.

In some embodiments, the stacking pad comprises a glass fiber tissue.

In some embodiments, the stacking pad has a width of 10 mm.

In some embodiments, the first length is 2 mm.

In some embodiments, the second length is 6 mm.

In some embodiments, the lateral flow assay device further comprises a nitrocellulose membrane, wherein the nitrocellulose membrane has a first end and a second end, the nitrocellulose membrane is overlapped by the conjugate pad by a first length at the first end, and the nitrocellulose membrane is overlapped by a wick pad by a second length at the second end.

In some embodiments, the wick pad comprises a chromatography paper.

In some embodiments, the wick pad is configured to display a result within 3 to 5 minutes of application of a sample to the sample pad.

In some embodiments, the nitrocellulose membrane comprises a glass fiber filter.

In some embodiments, the lateral flow assay device further comprises a detector antibody configured for conjugation on the conjugate pad.

In some embodiments, the conjugation is a short-scale conjugation.

In some embodiments, the detector antibody is selected from a mouse monoclonal IgG1 anti-benzodiazepine-BSA and/or a mouse monoclonal anti-ketamine-BSA.

In some embodiments, the stacking pad is pretreated with a Tris solution.

In some embodiments, the Tris solution has a concentration of at least 1 M.

In some embodiments, the Tris solution has a concentration of 1.3 to 1.7 M.

In some embodiments, the Tris solution has a concentration of about 1.5 M.

In some embodiments, the Tris solution further comprises at least 1% Tween 20 and has a pH of 8.5.

In some embodiments, the Tris solution is applied at a range of 2 to 8 mm from a base of the sample pad.

In some embodiments, the beverage is an alcoholic beverage.

In some embodiments, the alcoholic beverage is a beer.

In some embodiments, the alcoholic beverage is a seltzer.

In some embodiments, the stacking pad comprises a first layer and a second layer.

In some embodiments, the stacking pad has a basic pH.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a block diagram illustrating an exemplary test strip, according to embodiments of the present disclosure.

FIG. 2 is a block diagram illustrating an exemplary test strip with a stacking pad, according to embodiments of the present disclosure.

FIGS. 3A-C illustrate testing of stacking pad inclusion, according to embodiments of the present disclosure.

FIGS. 4A-B illustrate testing of stacking pad overlap, according to embodiments of the present disclosure.

FIGS. 5A-B illustrate testing of the short-scale conjugation method, according to embodiments of the present disclosure.

FIGS. 6A-B illustrate testing of the stacking pad material, according to embodiments of the present disclosure.

FIGS. 7A-B illustrate testing of the membrane materials, according to embodiments of the present disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

The present disclosure describes a multiplexed lateral flow assay for the qualitative detection of benzodiazepines and ketamine in alcoholic and non-alcoholic beverages. This assay provides consumers with a discreet, simple, and rapid method to detect the presence of common drugs of abuse, colloquially termed ‘date-rape drugs’, in social environments. In particular, the present disclosure describes a lateral assay flow test device with a series of overlapping pads to test alcoholic beverages, such as beers and alcoholic seltzers, for the presence of date-rape drugs such as benzodiazepines and ketamine.

Embodiments of the present disclosure relate to a testing device for collecting a sample and then testing it in situ, i.e., on the device, so that the outcome of the test is made available on the device to a user immediately or shortly after the collection of the sample. In particular, embodiments of the present disclosure relate to a lateral flow testing device, a testing device in which a sample collection element is in fluid communication via a flow path with one or more testing components.

Embodiments of the present disclosure describe a lateral flow assay device comprising a backing card with a plurality of pads along a length of the backing pad. The plurality of pads can include a sample pad, a conjugate pad, a stacking pad, a nitrocellulose membrane, and a wick pad. The pads can be oriented to cover the length of the backing card with some overlap between the pads. The inclusion of the stacking pad, which can enable faster conjugation of the sample deposited onto the sample pad, can result in a faster reading of the test strip device. In some embodiments, the stacking pad can comprise separate one or more materials to make up the entirety of the stacking pad itself. In some embodiments, the stacking pad can comprise a glass fiber.

A short-scale conjugation method can be used with the test strip device to optimize results. In some embodiments, the pH level of the conjugate is raised to have a more basic solution. In some embodiments, the short-scale conjugation method can improve manufacturability of the conjugate. In some embodiments, the short-scale conjugation improves the overall test line intensity and quality.

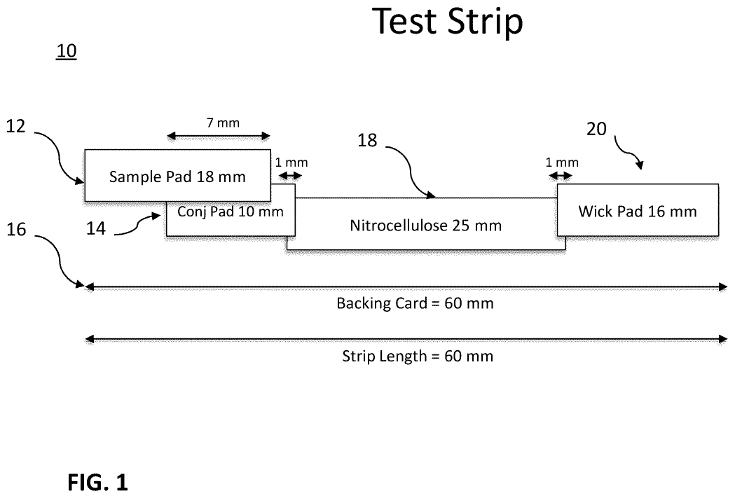

FIG. 1 is a block diagram of an exemplary embodiment of a sample test strip device 10. The test strip device 10 comprises a sample pad 12, a conjugate pad 14, a nitrocellulose membrane 18, and a wick pad 20, oriented lengthwise along the backing card 16. The sample pad 12 can comprise an absorbent material allowing for the sample to flow through the test strip device 10. For example, the sample pad 12 can comprise an 18 mm wide Fusion 5 strip, treated with 500 mM TBS, 1% Tween 20, pH 8.5 (Polarized pretreatment, 2-8 mm from base of sample pad), as outlined in Table 1, below. As further outlined in Table 1, the conjugate pad 14 can comprise a 10 mm wide Milipore GFDX pad treated with 10 mM NaPhos, 1% BSA, 1% Tween 20, pH 7.4 (Bath Method). The conjugate pad 14 can dispense conjugate at a rate of 1.5 L/mm, in some embodiments.

The backing card 16 runs the length of the test strip device 10. In the illustrated embodiment, the length of the test strip device 10 is 60 mm. However, the total length of the pads is greater than 60 mm, as certain pads overlap. To achieve the overlapping effect., the nitrocellulose membrane 18 is attached to the backing card 16, and each of the conjugate pad 14 and the wick pad 20 are then positioned such that an end of the conjugate pad 14 and wick pad 20 is attached to the nitrocellulose membrane. In the illustrated embodiment of FIG. 1, each of the conjugate pad 14 and the wick pad 20 overlap the nitrocellulose membrane 18 by only 1 mm. It is contemplated that this overlap can be larger or smaller, depending on desired size, testing materials, and other related parameters.

The sample pad 12 of the test strip device 10 has an end portion overlapping the conjugate pad 14. The sample pad 12, where samples of materials to be tested are deposited on the test strip device 10, is attached to the test strip device 10 such that, for example, 7 mm of the sample pad 12 lays along the conjugate pad 14. This can allow for easier fluid communication and quicker, more accurate results.

TABLE 1
Exemplary Parameters for Test Strip Device 10

| | | |
|---|---|---|
| CONJUGATE | Conjugate Pad Material | Millipore GFDX |
| PAD | Width | 10 mm |
| | Treatment | 10 mM NaPhos, 1% BSA, 1% Tween 20, pH 7.4 (Bath Method) |
| | Conjugate Dispense Rate | 1.5 μL/mm |
| SAMPLE | Sample Pad Material | Fusion 5 |
| PAD | Width | 18 mm |
| | Treatment | 500 mM TBS, 1% Tween 20, pH 8.5 (Polarized pretreatment, 2-8 |
| | | mm from base of sample pad) |
| CONJUGATE | Particle | 150 nm GNS [20 OD] |
| | Detector Antibody | BZD: mouse monoclonal IgG1 anti-benzodiazepine-BSA, |
| | | Meridian, Cat# G49651M |
| | | KET: mouse monoclonal anti-ketamine-BSA (LA280), East |
| | | Coast Bio, Cat# HM390 |
| | Antibody Purification | 1XPBS, Sigma Aldrich pH 7.4 Cat# P3813 |
| | Buffer | |
| | Antibody conc. | A₂₈₀ |
| | verification | |
| | Conjugation Tube | 1.5 mL Labcon tube; Labcon 15- or 50-mL conical tubes for |
| | | scale up |
| | Antibody Loading | 2 μg/mL (HM390); 4 ug/mL (G49651M) |
| | Antibody Incubation | 5 minutes |
| | Time | |
| | Reaction Buffer | 5 mM NaPhos, 0.5% PEG20, pH 7.4 |
| | Blocking | 4 mM sodium borate, 1% BSA, 0.05% sodium azide, pH 8.2 |
| | Blocking time | 30 minutes |
| | Final Diluent | 0.5x PBS, 0.5% casein, 0.5% BSA, 1% Tween 20, 0.05% sodium |
| | | azide, pH 8.0 |
| | Sugars (for drying down) | 10% Sucrose, 10% trehalose |
| | OD | 60 OD; G49651M at 60 OD, HM390 at 60 OD; Mixed 1:1 prior |
| | | to spraying |
| MEMBRANE | Type | Pall Vivid 120 (Part# VIV1202550R) |
| | Test Line Reagent | BZD: benzodiazepine-BGG, Meridian, Cat# Y01283B |
| | | KET: ketamine-BSA, East Coast Bio, Cat# P82-99-31A |
| | Test Line Concentration | 0.75 mg/mL [Ketamine-BSA, diluted in 1X PBS, Sigma] |
| | | 0.75 mg/mL [Benzodiazepine-BGG, diluted in 1X PBS, Sigma] |
| | Test Line Dispense Rate | 0.08 μL/mm |
| | Test Line Placement | BDZ: 10 mm from bottom edge of membrane (TL1; Pump 2) |
| | | KET: 5 mm from bottom edge of membrane (TL2; Pump 3) |
| | Control Line Reagent | Goat anti-mouse IgG, Lampire, Cat# 745507 |
| | Control Line | 1 mg/mL [1XPBS, Sigma] |
| | Concentration | |
| | Control Line Dispense | 0.08 μL/mm |
| | Rate | |
| | Control Line Placement | 15 mm from bottom edge of membrane (CL; Pump 1) |
| LF WICK | Material | Ahlstrom 222 |
| PAD | Width | 16 mm |
| SAMPLE | Beverage | Alcoholic or non-alcoholic drink |
| | Volume | 75 μL |
| FINAL LF | Cassette | KnoNap custom cassette |
| STRIP | Width | 3.8 mm |
| TESTING | Sample Volume | 75 μL |
| | Sample Chase Buffer | none |
| | Volume | |
| | Assay Eluent | none |
| | Conjugate Volume in Wet | N/A |
| | mode | |
| | Conjugate Chase Buffer | none |
| | Volume | |
| | Assay Run Time | 3-5 Minutes |

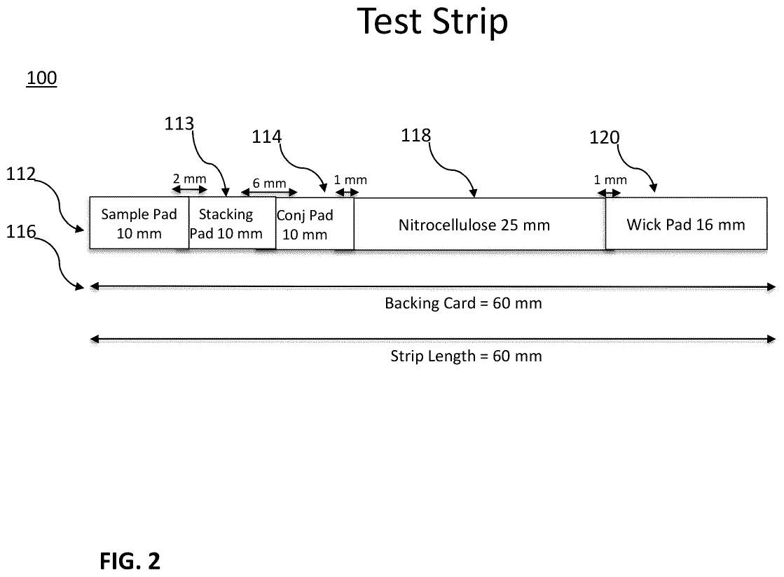

FIG. 2 is a block diagram of an exemplary embodiment of a sample test strip device 100 with a stacking pad 113. Similar to the test strip device 10 as shown in FIG. 1, The test strip device 100 comprises a sample pad 112, a conjugate pad 114, a nitrocellulose membrane 118, and a wick pad 120, oriented lengthwise along the backing card 116. However, the test strip device also includes stacking pad 113, which enables test strip device 100 to have a faster conjugation of the samples input into the test device. Exemplary parameters for the plurality of pads, conjugate, and testing device are further illustrated in Table 2, below.

In some embodiments, the stacking pad 113 can comprise a glass fiber. For example, in some embodiments, the stacking pad 113 comprises a 10 mm wide pad of Ahlstrom 8980, treated with 1 M Tris, 1% Tween 20, with pH 8.5. A polarized pre-treatment step can be used for the stacking pad, effectively removing any stacking issues within the test strip device 100. In some embodiments, the stacking pad 113 acts as a filter, and can aid in reducing the acidity of the samples. As test strip device 100 is designed to test primarily alcoholic beverages encountered within social settings, the stacking pad 113 can function to reduce the acidity of the sampled drinks, allowing for more accurate test results. The pretreatment of the stacking pad 113 can increase the pH to normalize each sample.

Much like the test strip device 10 as shown in FIG. 1, the backing card 116 runs the length of the test strip device 100. In the illustrated embodiment shown in FIG. 2, the length of the test strip device 100 is 60 mm. However, the total length of the pads is greater than 60 mm, as certain pads overlap. Here, the nitrocellulose membrane 118 is attached to the backing card 116, and each of the conjugate pad 114 and the wick pad 120 are then positioned such that an end of the conjugate pad 14 and wick pad 20 is attached to the nitrocellulose membrane. In the illustrated embodiment of FIG. 2, each of the conjugate pad 114 and the wick pad 120 overlap the nitrocellulose membrane 118 by only 1 mm. To allow space for the addition of stacking pad 113, one end of the stacking pad 113 is placed over and attached to the conjugate pad 114 for an exemplary overlap length of 6 mm. The sample pad 112 is then placed over the opposite second end of the stacking pad 113, for an exemplary overlap length of 2 mm as shown in FIG. 2.

The sample pad 112 of the test strip device 100 has an end portion overlapping the conjugate pad 114. The sample pad 112, where samples of materials to be tested are deposited on the test strip device 100, is attached to the test strip device 100 such that, for example, 2 mm of the sample pad 112 lays along the stacking pad 113.

TABLE 2
Exemplary Parameters for Test Strip Device 100

| | | |
|---|---|---|
| CONJUGATE | Conjugate Pad Material | Millipore GFDX |
| PAD | Width | 10 mm |
| | Treatment | 10 mM NaPhos, 1% BSA, 1% Tween 20, pH 7.4 (Bath Method) |
| | Conjugate Dispense Rate | 1.5 μL/mm |
| SAMPLE | Sample Pad Material | Fusion 5 |
| PAD | Width | 10 mm |
| | Treatment | 500 mM Tris, 1% Tween 20, pH 8.5 (Polarized pretreatment, 2-8 |
| | | mm from base of sample pad) |
| STACKING | Stacking Pad Material | Ahlstrom 8980 |
| PAD | Width | 10 mm |
| | Treatment | 1M Tris, 1% Tween 20, pH 8.5 (Polarized pretreatment, 2-8 mm |
| | | from base of sample pad) |
| CONJUGATE | Particle | 150 nm GNS [20 OD] |
| | Detector Antibody | BZD: mouse monoclonal anti-Benzodiazepine, Arista Biologicals, |
| | | Cat# 299.9 |
| | | KET: mouse monoclonal anti-ketamine-BSA (LA280), East |
| | | Coast Bio, Cat# HM390 |
| | | Co-Load: Goat IgG, Arista Biologicals, Cat#AGGIG-0100 |
| | Antibody Purification | 1XPBS, Sigma Aldrich pH 7.4 Cat# P3813 |
| | Buffer | |
| | Antibody conc. | A₂₈₀ |
| | verification | |
| | Conjugation Tube | 1.5 mL Labcon tube; Labcon 15- or 50-mL conical tubes for |
| | | scale up |
| | Antibody Loading | 3 μg/mL (HM390) + 27 ug/mL (Goat IgG); 1 ug/mL (299.9) + 24 |
| | | ug/mL (Goat IgG) |
| | Antibody Incubation | 5 minutes |
| | Time | |
| | Reaction Buffer | 50X PBS + 100 mM Borate, pH 8.0 (Ketamine); 50X PBS + 100 |
| | | mM NaPhos, pH 7.4 (Benzodiazepine) |
| | Blocking | 10% BSA (Fisher) in DI H₂O |
| | Blocking time | 15 minutes |
| | Final Diluent | 0.5x PBS, 0.5% casein, 0.5% BSA, 1% Tween 20, 0.05% sodium |
| | | azide, pH 8.0 |
| | Sugars (for drying down) | 10% Sucrose, 10% trehalose |
| | OD | OD 15 (299.9 - Benzodiazepine); OD 8 (HM390 - Ketamine) |
| MEMBRANE | Type | Pall Vivid 90 (Part# VIV1202550R) |
| | Test Line Reagent | BZD: benzodiazepine-BSA, Arista, Cat# AGBZO-0306 (Lot# |
| | | 022832210 for validation) |
| | | KET: ketamine-BSA, East Coast Bio, Cat# P82-99-31A |
| | Test Line Concentration | 0.75 mg/mL [Ketamine-BSA, diluted in 1X PBS, Sigma] |
| | | 0.75 mg/mL [Benzodiazepine-BGG, diluted in 1X PBS, Sigma] |
| | Test Line Dispense Rate | 0.08 μL/mm |
| | Test Line Placement | BDZ: 10 mm from bottom edge of membrane (TL1; Pump 2) |
| | | KET: 5 mm from bottom edge of membrane (TL2; Pump 3) |
| | Control Line Reagent | 40 nm Au-SA at 10 OD mixed with blue dye at 1:100 dilution |
| | Control Line Dispense | 0.08 μL/mm |
| | Rate | |
| | Control Line Placement | 15 mm from bottom edge of membrane (CL; Pump 1) |
| LF WICK | Material | Ahlstrom 222 |
| PAD | Width | 16 mm |
| SAMPLE | Beverage | Beers and Seltzers |
| | Volume | 75 μL |
| FINAL LF | Cassette | KnoNap custom cassette |
| STRIP | Width | 3.8 mm |
| TESTING | Sample Volume | 75 μL |
| | Sample Chase Buffer | none |
| | Volume | |
| | Assay Eluent | none |
| | Conjugate Volume in Wet | N/A |
| | mode | |
| | Conjugate Chase Buffer | none |
| | Volume | |
| | Assay Run Time | 3-5 Minutes |

### EXAMPLES

### Example 1

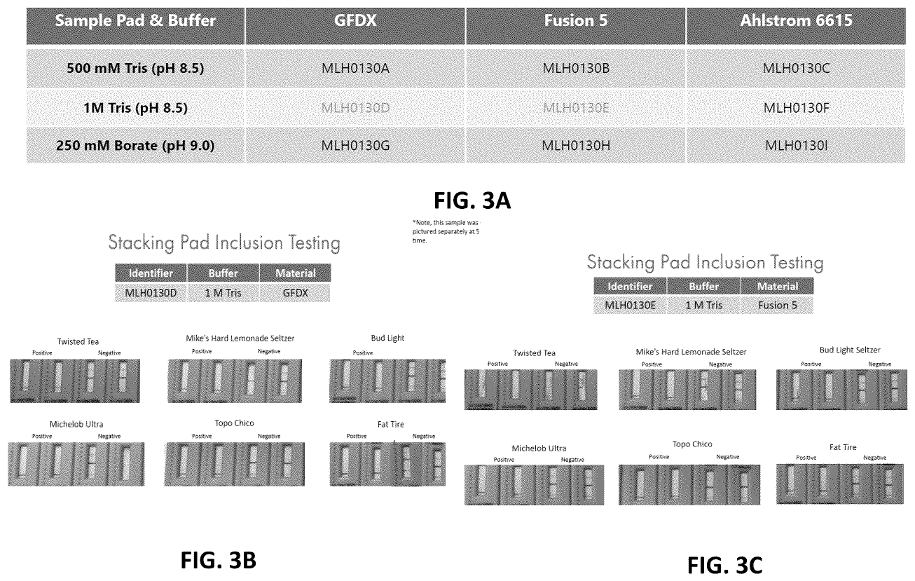

FIGS. 3A-C illustrate testing of stacking pad inclusion, according to embodiments of the present disclosure. The purpose of this testing is to separate the conjugate from the sample pad with an additional treated pad, intending to further treat the sample before it can aggregate the conjugates. The top row table, as shown in FIG. 3A, lists the different sample pad and buffer treatments. FIG. 3B and FIG. 3C show the resulting tests over a range of beers and alcoholic seltzers. The testing shown in FIG. 3B indicates that the inclusion of the stacking pad with the treatment significantly improved conjugate flow and result readability from an initial full sweep testing. The testing shown in FIG. 3C shows result improvement over initial testing, but liquid flow remains less consistent and clean in comparison to the samples tested in FIG. 3B.

### Example 2

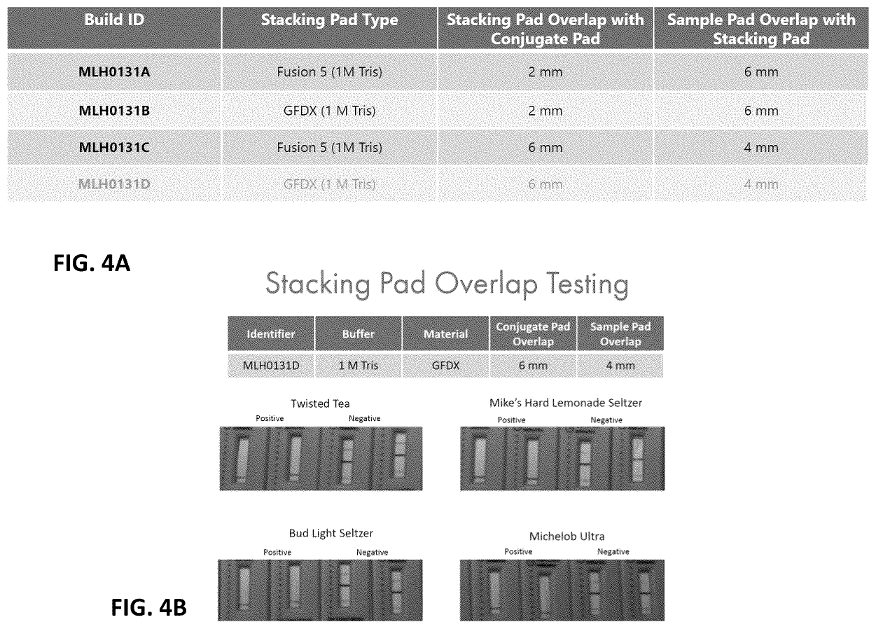

FIGS. 4A-B illustrate testing of stacking pad overlap, according to embodiments of the present disclosure. The purpose of this testing is to determine the best location on the test strip for the stacking pad when overlapping the sample and conjugate pads, using the parameters shown in the table of FIG. 4A. Build ID MLH0131D was identified as the best result. As shown in FIG. 4B, some streaking is still noticeable with some seltzers.

### Example 3

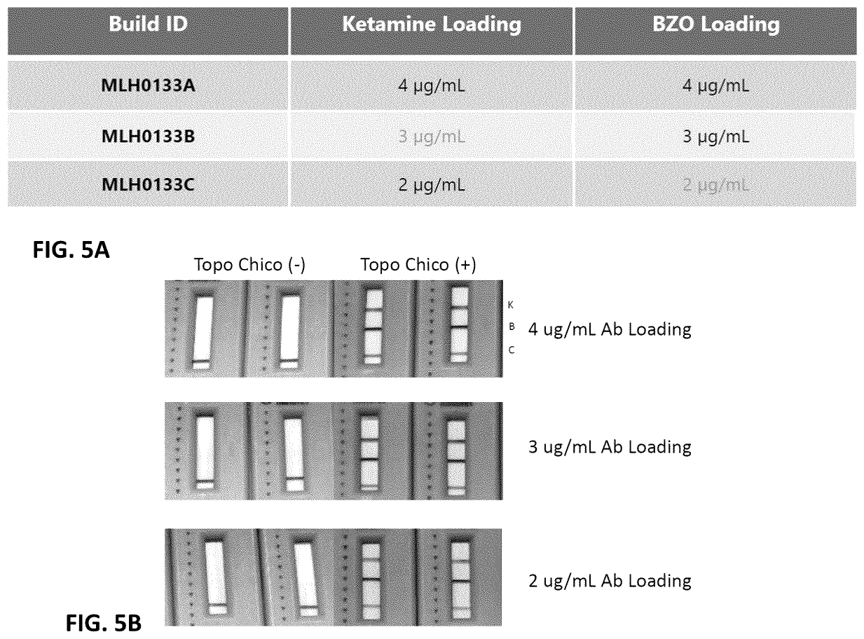

FIGS. 5A-B illustrate testing of the short-scale conjugation method, according to embodiments of the present disclosure. The purpose of this testing is to improve the manufacturability of the conjugate as well as to test the line intensity and quality, using the parameters shown in the table of FIG. 5A. The results in FIG. 5B are shown, with test line quality and intensity significantly improved with the transfer to the short-scale conjugation method.

### Example 4

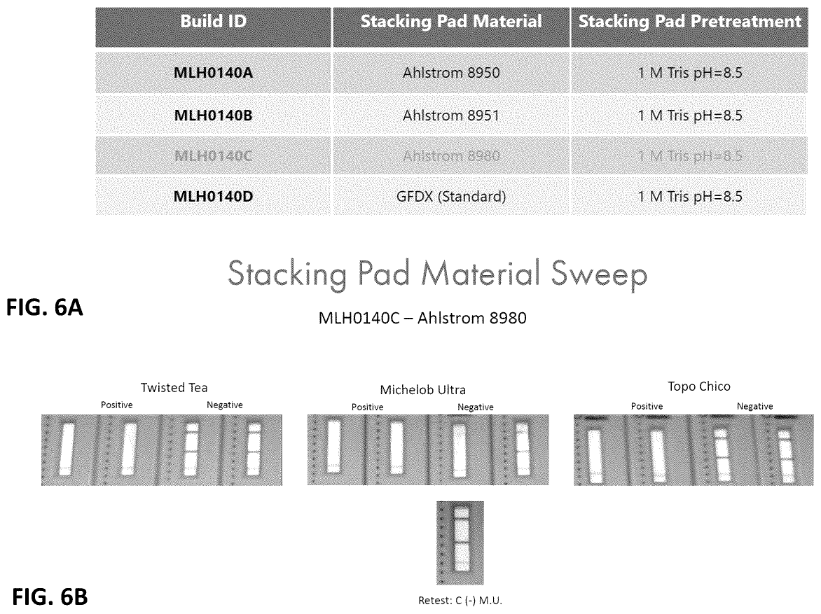

FIGS. 6A-B illustrate testing of the stacking pad material, according to embodiments of the present disclosure. The purpose of this testing is to evaluate other glass fiber-like stacking pads to determine any possible improvements to conjugate flow and sample treatment, using the parameters shown in FIG. 6A. The results are shown in FIG. 6B. Ahlstrom 8980 pretreated with 1 M Tris shows the best conjugate release and least aggregation compared to other stacking pads and will be used moving forward. This is notable for beverage testing, as concentrations of this strength generally “crashes” the fluid communication between the pads. If the sample input into the lateral flow assay is a bodily fluid, such as saliva or urine, the Tris concentration is held around 500 mM in order to achieve results. The added salt within a biofluid sample does not allow a conjugation reaction to occur, rendering the test unusable.

### Example 5

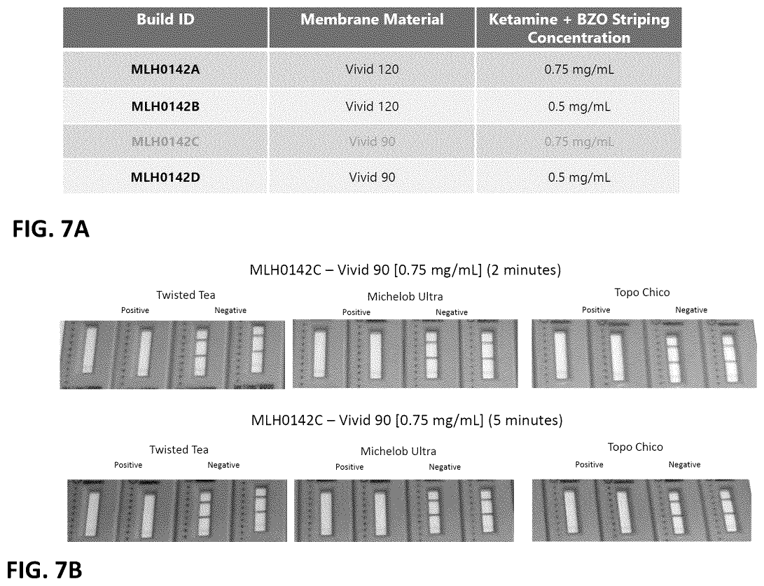

FIGS. 7A-B illustrate testing of the membrane materials, according to embodiments of the present disclosure. The purpose of this testing is to potentially reduce the total concentration of protein on the strip due to the current line intensity with the new conjugate method, and also look at a membrane with a quicker flow rate to improve test kinetics during the intended reading window, using the parameters shown in FIG. 7A. The results are shown in FIG. 7B. The Vivid 90 membrane at 0.75 mg/mL striping shows run times within desired window of 3-5 minutes while still maintaining acceptable line intensity.

What is claimed is:

1. A lateral flow assay device for testing beverages, the lateral flow assay device comprising:
a backing card;
a plurality of pads adhered to the backing card, the plurality of pads comprising at least a sample pad, a stacking pad, and a conjugate pad, wherein
the stacking pad has a first end and a second end, the stacking pad is overlapped by the sample pad by a first length at the first end, and the stacking pad overlaps the conjugate pad by a second length at the second end.

2. The lateral flow assay device of claim 1, wherein the plurality of pads forms a continuous flow path along the backing card.

3. The lateral flow assay device of claim 2, wherein each pad of the plurality of pads is in fluid communication with an overlapping pad of the plurality of pads.

4. The lateral flow assay device of claim 1, wherein the stacking pad comprises a glass fiber tissue.

5. The lateral flow assay device of claim 1, wherein the stacking pad has a width of 10 mm.

6. The lateral flow assay device of claim 1, wherein the first length is 2 mm.

7. The lateral flow assay device of claim 1, wherein the second length is 6 mm.

8. The lateral flow assay device of claim 1, further comprising a nitrocellulose membrane, wherein the nitrocellulose membrane has a first end and a second end, the nitrocellulose membrane is overlapped by the conjugate pad by a first length at the first end, and the nitrocellulose membrane is overlapped by a wick pad by a second length at the second end.

9. The lateral flow assay device of claim 8, wherein the wick pad comprises a chromatography paper.

10. The lateral flow assay device of claim 8, wherein the wick pad is configured to display a result within 3 to 5 minutes of application of a sample to the sample pad.

11. The lateral flow assay device of claim 8, wherein the nitrocellulose membrane comprises a glass fiber filter.

12. The lateral flow assay device of claim 1, further comprising a detector antibody configured for conjugation on the conjugate pad.

13. The lateral flow assay device of claim 12, wherein the conjugation is a short-scale conjugation.

14. The lateral flow assay device of claim 12, wherein the detector antibody is selected from a mouse monoclonal IgG1 anti-benzodiazepine-BSA and/or a mouse monoclonal anti-ketamine-BSA.

15. The lateral flow assay device of claim 1, wherein the stacking pad is pretreated with a Tris solution.

16. The lateral flow assay device of claim 15, wherein the Tris solution has a concentration of at least 1 M.

17. The lateral assay device of claim 15, wherein the Tris solution has a concentration of 1.3 to 1.7 M.

18. The lateral assay device of claim 15, wherein the Tris solution has a concentration of about 1.5 M.

19. The lateral flow assay device of claim 15, wherein the Tris solution further comprises at least 1% Tween 20 and has a pH of 8.5.

20. The lateral flow assay device of claim 15, wherein the Tris solution is applied at a range of 2 to 8 mm from a base of the sample pad.

21. The lateral flow assay device of claim 1, wherein the beverage is an alcoholic beverage.

22. The lateral flow assay device of claim 21, wherein the alcoholic beverage is a beer.

23. The lateral flow assay device of claim 21, wherein the alcoholic beverage is a seltzer.

24. The lateral flow assay device of claim 1, wherein the stacking pad comprises a first layer and a second layer.

25. The lateral flow assay device of claim 1, wherein the stacking pad has a basic pH.
